SEQUENCE LISTING (3 sequences)

SEQ ID NO: 1 - protein, 33 aa, Phyllomedusa bicolor
GLWSKIKEVGKEAAKAAAKAAGKAALGAVSEAV

SEQ ID NO: 2 - protein, 81 aa, Phyllomedusa bicolor
MAFLKKSLFLVLFLGLVSLSICEEEKRENEDEEEQEDDEQSEMKRGLWSKIKEVGKEAAKAAAKAAGKAALGAVSEAVGEQ

SEQ ID NO: 3 - DNA, 380 bp, Phyllomedusa bicolor
tccggcactttctgaattacaagaccagacatggctttccttaagaaatctcttttccttgtactattccttggattggtctctctttctatctgcgaagaagagaaaagagaaaatgaagatgaggaggaacaagaagatgacgagcaaagtgaaatgaagagagggctttggagtaaaataaaagaagtaggaaaagaagcagcaaaagctgcagcaaaagctgcaggaaaagcggctttaggtgcagtttctgaggcggtgggagagcaataaatataagaaaatgtaaaatcaaattgctcttaacagagcaattatcaatatttgtgccaaacctttattaaagcaaattgaactgaaaaaaaaaaaaaaaaaaa

(19) United States
(12) Patent Application Publication
(10) Pub. No.: US 2012/0184498 A1
(43) Pub. Date: Jul. 19, 2012

(54) DERMASEPTIN B2 USED AS AN INHIBITOR OF THE GROWTH OF A TUMOR

(75) Inventors: Jean Delbe, Ville D'avray (FR); Mohamed Amiche, Noisiel (FR); Ali Ladram, Ermont (FR); Cécile Galanth, Paris (FR); Pierre Nicolas, Villez-sous-Bailleul (FR); Yamina Hamma, Le Kremlin Bicetre (FR); Johanna Allegonda Anna Van Zoggel, Vitry Sur Seine (FR); José Courty, Villecresnes (FR)

(73) Assignees: UNIVERSITÉ PARIS EST CRETEIL VAL DE MARNE, Creteil Cedex (FR); UNIVERSITÉ PIERRE ET MARIE CURIE (PARIS 6), Paris (FR)

(21) Appl. No.: 13/381,477

(22) PCT Filed: Jun. 29, 2010
(86) PCT No.: PCT/FR10/51347

§ 371 Date: Apr. 2, 2012

(30) Foreign Application Priority Data
Jul. 1, 2009 (FR) 0954505

(51) Int. Cl. A61K 38/17; C12N 15/63; A61K 31/7088; A61P 19/02; C07H 21/04; A61P 35/00; A61P 35/02; A61P 35/04; C12N 9/96; C07K 14/435

(52) U.S. Cl. 514/21.2; 435/188; 435/320.1; 514/21.3; 514/44 R; 530/324; 536/23.5

(57) ABSTRACT

The invention relates to the use of peptides corresponding to dermaseptin B2 or fragments thereof for treating proliferative diseases such as cancer or ocular lesions, and to pharmaceutical compositions containing such peptides.

The present invention concerns the use of dermaseptin B2 to inhibit cell proliferation and/or growth.

Current cancer therapy is based on radiotherapy, surgery, at times most invalidating, and/or the use of anticancer drugs to block mitosis. These therapies are often very aggressive which may limit the use thereof. Another envisaged treatment pathway is immunotherapy which consists in administering substances which will stimulate the immune defenses of the organism. Nevertheless, patients suffering from cancer, metastatic cancer in particular, do not always respond or respond poorly to immunotherapy. Despite major research over the world, there is currently no universal therapy against this pathology.

In addition to uncontrolled proliferation of tumour cells, the long-term development of a tumour is always associated with angiogenesis. Therefore, the suppression, or the inhibition of factors inducing angiogenesis must lead to the regression of tumour growth irrespective of the type of tumour.

At the current time several companies, for the treatment of cancer or proliferative diseases, are developing an anti-angiogenesis strategy based for example on inhibitors of angiogenic factors (or of their receptors when these are identified) or by inducing vascular micro-thromboses using the endothelial cell as anchor for the catalysts of thrombosis, or using peptide agents which inhibit angiogenesis via mechanisms that are not always identified. These approaches have not yet led to clear results, and it appears that the inhibitors based on blocking a single angiogenesis pathway induce aggravating rebound effects. These results have led these companies to proposing cocktails of inhibitors enabling hoping obtaining radical and simultaneous destruction of vessels and tumour cells.

There is therefore a need at the present time for novel inhibitors of angiogenesis which, on this account, inhibit the growth and/or proliferation of cells.

The inventors have now shown that surprisingly dermaseptin B2, extracted from the skin secretions of the South American frog of genus Phyllomedusa bicolor and known as anti-microbial agent, is capable of inhibiting firstly the proliferation of tumour cells and secondly angiogenesis.

American patent U.S. Pat. No. 6,440,690 describes the use of peptides, in particular of dermaseptin for the treatment of infectious diseases or cancer, by stimulating the host's immune system. More precisely, these peptides stimulate the immune system by activating the cells of monocyte/macrophage cell lines and/or other lymphoid cells. On the other hand, it is not suggested that dermaseptin B2 could inhibit cell growth and/or proliferation.

### DISCLOSURE OF THE INVENTION

The present invention therefore concerns an isolated peptide comprising or consisting in a sequence of amino acids selected from the group consisting of:
the sequence of dermaseptin B2, the sequence of the precursor of dermaseptin B2;
a sequence of amino acids having at least 80% identity with the sequences of dermaseptin B2 or of the precursor of dermaseptin B2; and
a fragment of these sequences,

provided that the isolated peptide inhibits cell growth and/or proliferation, for use, preferably to inhibit cell growth and/or proliferation, in the treatment or prevention of a proliferative disorder, an ocular lesion or an auto-immune disease.

The present invention also concerns a chimeric molecule comprising at least one peptide as defined above, wherein said peptide is linked with:
a) a therapeutic compound useful for the treatment of proliferative disorders;
b) an enzyme capable of converting a molecule into a therapeutic compound useful for the treatment of proliferative disorders; or
c) a carrier molecule.

The present invention further concerns a method for producing a chimeric molecule as defined above, comprising:
a) the synthesis of a peptide as defined above via chemical route; and
b) conjugation of said peptide with a compound selected from:
   i) a therapeutic compound useful for the treatment of proliferative disorders;
   ii) an enzyme capable of converting a molecule into a therapeutic compound useful for the treatment of proliferative disorders; and
   iii) a carrier protein.

A further subject of the present invention concerns a nucleic acid comprising or consisting of a sequence coding for a peptide as defined above, and a vector comprising this nucleic acid in which the nucleic acid is functionally linked to one or more elements allowing the expression of said peptide, for use, preferably to inhibit cell growth and/or proliferation, in the treatment or prevention of a proliferative disorder, an ocular lesion or an auto-immune disease.

The present application also describes a pharmaceutical composition comprising a peptide as defined above or a chimeric molecule as defined above, and a pharmaceutical composition comprising a nucleic acid as defined above or a vector as defined above. These compositions may further comprise a second therapeutic compound useful for the treatment of proliferative disorders, ocular lesions or auto-immune diseases.

### Dermaseptin 82

By “dermaseptin B2” or “adenoregulin” is meant herein a peptide of 33 amino acids which was isolated for the first time from skin secretions of a South American frog of genus Phyllomedusa bicolor (Daly et al. (1992) Proc. Natl. Acad. Sci. USA 89:10960-10963). The amino acid sequence of this peptide corresponds to the sequence GLWSKIKEVGKEAAKAAAKAAGKAALGAVSEAV (SEQ ID NO: 1). This peptide is expressed in the form of a precursor, preproadenoregulin, which consists of 81 amino acids and whose sequence is the following:

maflkkslflvlflglvslsiceeekrenedeeeqeddeqsemkrglwskikevgkeaakaaakaagkaalgaysea vgeq (SEQ ID NO: 2) (Amiche et al. (1994) J. Biol. Chem. 269:17847-17852). The cDNA corresponding to this precursor was identified and is shown in sequence SEQ ID NO: 3.

Dermaseptin B2 is known to increase the binding of agonists with the receptor of adenosin Al (Daly et al. (1992) Proc. Natl. Acad. Sci. USA 89:10960-10963; Moni et al. (1995) Cell. Mol. Neurobiol. 15:465-493). A structural and pharmacological analysis has shown that it is related to the large family of dermaseptins, which are broad spectrum antimicrobial peptides isolated from Amazonian tree frogs (Amiche et al. (1994) J. Biol. Chem. 269:17847-17852; Charpentier et al. (1998) J. Biol. Chem. 273:14690-14697; Mor et al. (1994) J. Biol. Chem. 269:31635-31641; Mor et al. (1991) Biochemistry 30:8824-8830). The solid phase chemical synthesis of this peptide does not offer any particular difficulty since it is of relatively short size and its structure is not or only scarcely deteriorated by post-translational modifications. In addition, its production via recombinant expression in Escherichia coli is also possible on account of the availability of its cDNA.

As is well known to persons skilled in the art, dermaseptin B2 in micromolar doses rapidly kills Gram+ and Gram− bacteria, yeasts, protozoa and filamentous fungi. In addition it is devoid of haemolytic activity.

### Peptides

The peptides of the invention have biological activity. By “biological activity” it is particularly meant herein activity inhibiting angiogenesis and/or cell proliferation and/or tumour growth. A peptide of the invention has biological activity as soon as it has at least one of the above-mentioned activities. Preferably the peptides have an inhibiting activity of cell proliferation and/or cell growth, in particular of tumour or vascular cells.

The cell proliferation and/or of tumour growth inhibiting activity of a peptide can easily be assessed in vitro or in vivo, by persons skilled in the art, in particular by means of the following assays:
in vitro, by (i) contacting the peptides with cells of fibroblast type (e.g. NIH 3T3) stimulated by a growth factor, in the absence of serum, (ii) addition of thymidine labeled with radioactivity and (iii) measurement of the radioactivity incorporated by the cells, for example using liquid scintillation;
in vitro, by contacting the peptides with tumour cells (e.g. PC-3) in soft agar, and observation of cell growth by measurement of their diameter;
in vivo, by injection of peptides into nude mice in which tumours were induced by injection of PC-3, and observation of tumour growth by measurement of the volume and/or weight of the tumours.

By “isolated” peptide is meant herein a peptide isolated from the organism of an animal or microorganism. However, the isolated peptide may be present for example in a pharmaceutical composition or a kit. Preferably, the peptide is present in one of the pharmaceutical compositions described below. This peptide is preferably in purified form. The peptide of the invention can be synthesized via chemical or biological route. In particular it may be recombinantly produced.

Preferably, the peptide of the invention has a size of between 6 and 81, 6 and 76, 6 and 71, 6 and 66, 6 and 61, 6 and 56, 6 and 51, 6 and 46, 6 and 41, 6 and 36, 6 and 33, 6 and 30, 6 and 28, 6 and 26, 6 and 24, 6 and 22, 6 and 20, 6 and 18, 6 and 16, 6 and 14, 6 and 12, 6 and 10 amino acids, preferably a size of 9, 8 or 7 amino acids. Most preferably, the peptide of the invention has a size of 33 amino acids.

The said peptide may in particular comprise or consist of a sequence of amino acids selected from the group consisting of sequences SEQ ID NO: 1 and SEQ ID NO: 2; an amino acid sequence having at least 80% identity with sequences SEQ ID NO: 1 or SEQ ID NO: 2; and a fragment of these sequences provided that the isolated peptide inhibits cell growth and/or proliferation. Preferably, said peptide consists of a sequence of amino acids selected from the group consisting of sequences SEQ ID NO: 1 and SEQ ID NO: 2.

The said peptide may also consist of a fragment of the sequences SEQ ID NO: 1 or SEQ ID NO: 2.

By “fragment” of a reference sequence is meant herein a sequence constituted by a chain of consecutive amino acids of a reference sequence and whose size is smaller than the size of the reference sequence. In the context of the invention, the fragments may for example have a size of between 6 and 76, 6 and 71, 6 and 66, 6 and 61, 6 and 56, 6 and 51, 6 and 46, 6 and 41, 6 and 36, 6 and 33, 6 and 30, 6 and 28, 6 and 26, 6 and 24, 6 and 22, 6 and 20, 6 and 18, 6 and 16, 6 and 14, 6 and 12, 6 and 10 amino acids, preferably a size of 9, 8 or 7 amino acids. In particular, the fragments may have a size of 33 amino acids. Preferably these fragments are derived from the C-terminal end of dermaseptin B2 or of the precursor of dermaseptin B2.

The peptides of the invention also include peptides having sequences derived from sequence SEQ ID NO: 1 or SEQ ID NO: 2, or derived from fragments of sequence SEQ ID NO: 1 or SEQ ID NO: 2, defined by percentage sequence identity with one of sequences SEQ ID NO: 1 or SEQ ID NO: 2. These derived sequences may differ from the reference sequence by substitution, deletion and/or insertion of one or more amino acids, at positions such that these modifications do not have any significant impact on the biological activity of the peptides. The substitutions may in particular correspond to conservative substitutions or to substitutions of natural amino acids by non-natural amino acids or pseudo amino acids.

By “amino acid sequence having at least 80% (for example) sequence identity with a reference sequence” is meant herein a sequence identical to the reference sequence but this sequence may comprise up to twenty mutations (substitutions, deletions and/or insertions) per each part of one hundred amino acids of the reference sequence. Therefore for a reference sequence of 100 amino acids, a fragment of 80 amino acids and a sequence of 100 amino acids comprising 20 substitutions compared with the reference sequence are two examples of sequences having 80% sequence identity with the reference sequence.

Percentage identity is generally determined using sequence analysis software (for example the Sequence Analysis Software Package of the Genetics Computer Group, University of Wisconsin Biotechnology Center, 1710 University Avenue, Madison, Wis. 53705). The amino acid sequences to be compared are aligned to obtain maximum percentage identity. For this purpose, it may be necessary to artificially add gaps in the sequence. The alignment can be performed manually or automatically. Automated alignment algorithms of nucleotide sequences are well known to persons skilled in the art and described for example in Altschul et al. (1997) Nucleic Acids Res. 25:3389 and implemented by softwares such as the Blast software. One algorithm which can be isolated is the Needleman-Wunsch algorithm for example (Needleman and Wunsch (1970) J Mol Biol. 48:443-53). Once optimal alignment has been achieved, the percentage identity is established by recording all the positions at which the amino acids of the two compared sequences are identical, compared with the total number of positions.

Therefore, the peptides of the invention may comprise or consist of a sequence selected from:
a fragment of a sequence having at least 80%, 85%, 90%, 95% or 100% sequence identity with SEQ ID NO: 1 or SEQ ID NO : 2; and
a sequence having at least 80%, 85%, 90%, 95% or 100% sequence identity with sequence SEQ ID NO: 1 or SEQ ID NO: 2.

In one particular embodiment, the sequence of the peptides differs from sequence SEQ ID NO: 1 or SEQ ID NO: 2, or from a fragment of sequence SEQ ID NO: 1 or SEQ ID NO: 2, solely through the presence of conservative substitutions. Conservative substitutions are substitutions of amino acids of the same class, such as substitutions of amino acids with non-charged side chains (such as asparagine, glutamine, serine, cysteine, and tyrosine), of amino acids with basic side chains (such as lysine, arginine and histidine), of amino acids with acid side chains (such as aspartic acid and glutamic acid), of amino acids with non-polar side chains (such as alanine, valine, leucine, isoleucine, proline, phenylalanine, methionine and tryptophan).

According to the invention, the peptides may be modified chemically or enzymatically to improve their stability or bioavailability. Such chemical or enzymatic modifications are well known to those skilled in the art. Mention may be made of the following modifications but they are not limited thereto:
modifications of the C-terminal or N-terminal end of the peptides such as N-terminal deamination or acylation (preferably acetylation) or such as C-terminal amidation or esterification;
modifications of the amide bond between two amino acids, such as acylation (preferably acteylation) or alkylation at the nitrogen or alpha carbon;
changes in chirality, such as the substitution of a natural amino acid (L-enanthiomer) by the corresponding D-enanthiomer. This modification may optionally be accompanied by inversion of the side chain (from the C-terminal end to the N-terminal end);
changes to azapeptides, in which one or more alpha carbons are replaced by nitrogen atoms; and/or
changes to betapeptides, in which one or more carbons are added on the N-alpha side or on the C-alpha side of the main chain.

In this respect, it is possible to modify one or more of the lysine amino acids (K) of the peptides, notably by:
amidation: this modification is simple to achieve, the positive charge of the lysine being substituted by hydrophobic groups (for example acetyl or phenylacetyl);
amination: by formation of secondary amide from the primary amine R=(CH₂)₄—NH₃⁺, for example by forming N-methyl, N-allyl or N-benzyl groups; and
by formation of N-oxide, N-nitroso, N-dialkyl phosphoryl, N-sulfenyl, or N-glycoside groups.

It is also or alternatively possible to modify one or more threonine (T) and/or serine (S) amino acids of the peptides, notably by adding an ester or ether group at the OH group of the side chain of threonine and/or serine. Esterification, a simple operation, can be performed using a carboxylic acid, an anhydride, by bridging, etc, to form acetates or benzoates. Etherification, which gives more stable compounds, can be performed using an alcohol, a halide, etc. to form a methyl ether for example or an O-glycoside.

It is also or alternatively possible to modify one or more glutamine (Q) amino acids for example by amidation, by forming secondary or tertiary amines, in particular with groups of methyl, ethyl type, whether or not functionalized.

It is also or alternatively possible to modify one or more glutamate (E) and/or aspartate (D) amino acids, for example:
by esterification, to form methyl esters, whether or not substituted, ethyl esters, benzyl esters, thiols (activated esters); and
by amidation, notably to form N,N dimethyl groups, nitroanilides, pyrrolidinyls.

On the other hand, it is preferable not to modify the proline amino acids, which take part in the secondary structure of the peptides, bearing also in mind that the amino acids G, A and M in general do not offer modification possibilities of clear interest.

### Chimeric molecule

The invention also concerns a chimeric molecule comprising at least a peptide according to the invention as defined above, wherein said peptide is linked to:
a) a therapeutic compound useful for the treatment of proliferative disorders;
b) an enzyme capable of converting a molecule into a therapeutic compound useful for the treatment of proliferative disorders; or
c) a carrier molecule.

By “chimeric molecule” is meant herein a molecule comprising or consisting of a peptide according to the invention linked to another molecule. The peptide of the invention is linked to the other molecule via a covalent bond. The bond preferably corresponds to chemical coupling. However, when the molecule with which the peptide is linked is another polypeptide, the two peptides can be in the form of a fusion protein.

By “carrier molecule” is meant herein any molecule with which at least one peptide can be coupled (conjugated). Preferably, the carrier molecule is of sufficient size so that it can be coupled with at least 3 peptides according to the invention, preferably 3 to 8 peptides of the invention. In the meaning of the invention, by “coupled”, for a peptide, is meant the fact that it is linked to the carrier molecule via a covalent bond, either directly or via a spacer compound between the peptide and the carrier molecule. Examples of acceptable spacers comprise compounds of the type ethylene glycol, piperazine, or an amino acid of aminohexanoIc acid or beta-alanine type.

Persons skilled in the art know such carrier molecules with which a peptide can advantageously be coupled.

For example, peptides are commonly coupled with Keyhole Limpet Hemocyanin (KLH), bovine serum albumin (BSA), ovalbumin (OVA), thyroglobulin (THY) or MAP (multiple antigenic peptide).

In one preferred embodiment, the carrier molecule corresponds to a support such as described in the PCT application published under number WO 2007/125210. Such a support may notably be selected from a linear peptide or a cyclic peptide, a linear or cyclic peptoid (oligomer of N-substituted glycine) a foldamer (oligomer or polymer having a strong tendency to assume a predictable, compact, well defined conformation in solution), a linear polymer or a spherical dendrimer (macromolecule constituted by monomers which group together as per a highly-branched process around a central pluri-functional core), a sugar, or a nanoparticle. Advantageously, the said support is selected from a linear or cyclic peptide, or a linear or cyclic peptoid. The use of a linear peptide allows easy synthesis of the support. A linear peptide acting as support in the invention may advantageously comprise a proportion of lysine of more than 25%. More precisely, when a linear peptide is used as support in the invention, the peptide(s) of the invention are preferably grafted on a lysine. If the support is a linear or cyclic peptide and if the peptide(s) of the invention are grafted directly onto the peptide, the bond between the supporting peptide and the peptide(s) of the invention is preferably formed at a lysine residue of the supporting peptide, at an amino group at position a or E, preferably at the amino group at position E (on the side chain) of the lysine. Therefore, the direct grafting of the peptide(s) of the invention onto a peptidic support is advantageously performed via an amide bond between an acid COOH function of the amino acid at the C-terminal end of the pseudo-peptide motif and an amino group of a lysine residue, preferably the amino group at position E (on the side chain) of the lysine. Advantageously the support of a peptide of the invention is selected from a cyclic hexapeptide constituted alternately of alanine residues (A) of configuration D and lysine residues (K) of configuration L.

The chimeric molecule of the invention may also comprise at least one peptide of the invention linked to an enzyme or to a therapeutic compound useful for the treatment of proliferative disorders.

In one preferred embodiment, the peptide of the invention is linked to a cytotoxic compound.

Alternatively, the peptide of the invention may be linked to an enzyme capable of converting a pro-drug into a therapeutic compound useful for treating proliferative disorders (see for example patents U.S. Pat. No. 5,760,072 and U.S. Pat. No. 5,433,955).

The peptide of the invention may for example be linked to an enzyme present in the matrix environment such as members of the matrix metalloproteinase family, urokinase or plasmin.

The peptide of the invention may for example be linked to a therapeutic compound selected from the group consisting of an N-terminal segment of human annexin 1, anti-inflammatory cytokines (in particular IL10 and IL13), non-activating inhibitors of the membrane receptors of pro-inflammatory cytokines, glucocorticoids, non-steroid anti-inflammatories and methotrexate.

The peptide of the invention may be linked to the therapeutic compound via a linker which is recognized and cleaved by an enzyme or a group of enzymes specific to the environment of cancer cells. More particularly, this enzyme can be selected from a metalloprotease of the extracellular matrix, a urokinase, and a protease specific for cleaving the extracellular segment of the membrane cytokines or of their receptors. The cleaving of the linker at the cancer cells then allows the release of two active ingredients; the peptide of the invention firstly and the therapeutic compound secondly.

Such chimeric molecules can be used as medicament, more particularly for the treatment or the prevention of a proliferative disorder, of an ocular lesion and/or an auto-immune disease. Preferably, such chimeric molecules are used in the treatment or prevention of a proliferative disorder such as cancer.

### Therapeutic Use

The peptides of the invention have inhibiting properties of cell proliferation and/or of cell growth. In this respect, these peptides and the chimeric molecules comprising them are particularly useful for the treatment of various pathologies associated with cell proliferation and growth.

One aspect of the invention therefore concerns a peptide or a chimeric molecule according to the invention for use in the treatment or prevention of a proliferative disorder, of an ocular lesion and/or of an auto-immune disease, in particular to inhibit cell growth and/or proliferation.

By “proliferative disorder”, is meant herein any abnormal proliferation of cells, whether benign or malignant (cancerous). The peptides of the invention are particularly useful for the treatment and/or prevention of cancers.

Proliferative disorders notably include tumours. The invention more particularly concerns cancer tumours, whether or not they are solid. The invention concerns the treatment and/or prevention of solid tumours such as melanomas, carcinomas, sarcomas, rhabdomyosarcoma, retinoblastoma, neuroblastoma, osteosarcoma, glioblastoma, mammary and ovarian tumours (whether or not primitive), lung tumours, tumours of the cervix, of the digestive tract in particular of the colon, of the urologic system, of the liver, pancreas, bones. Nonsolid tumours are also concerned, namely leukaemia or lymphomas in particular. Again with reference to cancerous tumours, the peptides of the invention are particularly useful for treating tumour metastases and/or for the prevention of the formation thereof. Amongst the benign tumours, also concerned by the present invention, mention may be made of haemangioma and hepatocellular adenomas.

Proliferative disorders also include disorders other than tumours, such as rheumatoid arthritis (RA) which is an inflammatory disease associated with intense angiogenesis, and skin diseases such as psoriasis. In this pathology there is proliferation of the synoviacytes, which is on the basis of the creation of inflammatory pannus. The mechanisms associated with this type of pathology look like mechanisms which lead to tumour growth.

“Ocular lesions” notably include pathologies of the retina such as diabetic retinopathy, macular degeneration, renal vein or artery occlusion, glaucoma. The peptides may also be useful for treating ocular lesions which may be consequence of reparative surgery such as corneal graft. In particular there is one of form of macular degeneration that is age-related said to be “exudative”. This type of pathology leads to the formation of abnormal blood vessels underneath the retina. This uncontrolled increase in vessels may over the longer term damage the macula and lead to blindness. Regarding diabetic retinopathy, this pathology is a disease of the retinal capillaries which become abnormal with notably disappearance of the pericytes. Rupture of the blood-retinal barrier is then observed leading to vascular hyperpermeability. These deteriorations will lead to retinal ischemia causing neo-angiogenesis which over the long term will lead to blindness. In these types of pathologies, etiopathology is based on the development of an uncontrolled vascular network. Therefore the use of anti-angiogenic molecules such as the peptides of the invention is an effective therapeutic treatment against these pathologies.

Since the peptides of the invention have anti-angiogenic properties, they are useful for treating or preventing auto-immune diseases (Griffioen et al. (1999) Int J Cancer. 80:315-9; Griffioen (2008) Cancer Immunol Immunother. 57:1553-8). “Auto-immune diseases” particularly include multiple sclerosis (MS), inflammatory bowel disease (IBD) in particular Crohn's disease, and lupus erythematosus.

Finally, the peptides and chimeric molecules of the invention may also be useful as abortive compounds for birth control by blocking uterine angiogenesis and hence embryo implantation.

Proliferative disorders can be treated at any stage of proliferation. By “treatment” is meant curative treatment (intended at least to relieve, slow or stop the development of the pathology). By “prevention” is meant prophylactic treatment (intended to reduce the risk of onset of the pathology).

The peptides and chimeric molecules of the invention may also be used in combination with a second active ingredient intended to treat or prevent the same disease. In the context of the treatment and/or prevention of cancers, the peptides can be used for example in combination with surgery for the removal of tumours, radiotherapy, chemotherapy, hormonotherapy, and/or immunotherapy.

A further subject of the present invention is therefore a peptide or chimeric molecule according to the invention, in combination with at least one therapeutic compound useful for the treatment of proliferative disorders, ocular lesions or auto-immune diseases, for use in the treatment and/or prevention of a proliferative disorder, an ocular lesion or an auto-immune disease.

By “therapeutic compound useful for the treatment of proliferative disorders, of ocular lesions or of auto-immune diseases” is meant any active ingredient other than the peptides of the invention which is useful for treating and/or preventing proliferative disorders, ocular lesions or auto-immune diseases. For example, it is possible to combine the peptides of the invention with an active ingredient already having marketing authorization and intended to treat these diseases. Said therapeutic compounds useful for treating proliferative disorders particular include the following compounds which have already been approved for treating various cancers: Abraxane, Adriamycin (Doxorubicin Hydrochloride), Adrucil (Fluorouracil), Aldara (Imiquimod), Alemtuzumab, Alimta, Pemetrexed Disodium), Aminolevulinic Acid, Anastrozole, Aprepitant, Arimidex (Anastrozole), Aromasin (Exemestane), Arranon (Nelarabine), Arsenic Trioxide, Avastin (Bevacizumab), Azacitidine, Bendamustine Hydrochloride, Bevacizumab, Bexarotene, Bexxar (Tositumomab and I 131 Iodine Tositumomab), Bortezomib, Campath (Alemtuzumab), Camptosar (Irinotecan Hydrochloride), Capecitabine, Carboplatin, Cetuximab, Cisplatin, Clafen (Cyclophosphamide), Clofarabine, Clofarex (Clofarabine), Clolar (Clofarabine), Cyclophosphamide, Cytarabine, Cytosar-U (Cytarabine), Cytoxan (Cyclophosphamide), Dacogen (Decitabine), Dasatinib, Decitabine, DepoCyt (Liposomal Cytarabine), DepoFoam (Liposomal Cytarabine), Dexrazoxane Hydrochloride, Docetaxel, Doxil (Doxorubicin Hydrochloride Liposome), Doxorubicin Hydrochloride, Doxorubicin Hydrochloride Liposome, Dox-SL (Doxorubicin Hydrochloride Liposome), Efudex (Fluorouracil), Ellence (Epirubicin Hydrochloride), Eloxatin (Oxaliplatin), Emend (Aprepitant), Epirubicin Hydrochloride, Erbitux (Cetuximab), Erlotinib Hydrochloride, Evacet (Doxorubicin Hydrochloride Liposome), Evista (Raloxifene Hydrochloride), Exemestane, Faslodex (Fulvestrant), Femara (Letrozole), Fluoroplex (Fluorouracil), Fluorouracil, Fulvestrant, Gefitinib, Gemcitabine Hydrochloride, Gemtuzumab Ozogamicin, Gemzar (Gemcitabine Hydrochloride), Gleevec (Imatinib Mesylate), Herceptin (Trastuzumab), Hycamtin (Topotecan Hydrochloride), Ibritumomab Tiuxetan, Imatinib Mesylate, Imiquimod, Iressa (Gefitinib), Irinotecan Hydrochloride, Ixabepilone, Ixempra (Ixabepilone), Keoxifene (Raloxifene Hydrochloride), Kepivance (Palifermin), Lapatinib Ditosylate, Lenalidomide, Letrozole, Levulan (Aminolevulinic Acid), LipoDox (Doxorubicin Hydrochloride Liposome), Liposomal Cytarabine, Methazolastone (Temozolomide), Mylosar (Azacitidine), Mylotarg (Gemtuzumab Ozogamicin), Nanoparticle Paclitaxel (Paclitaxel Albumin-stabilized Nanoparticle Formulation), Nelarabine, Neosar (Cyclophosphamide), Nexavar (Sorafenib Tosylate), Nilotinib, Nolvadex (Tamoxifen Citrate), Oncaspar (Pegaspargase), Oxaliplatin, Paclitaxel, Paclitaxel Albumin-stabilized Nanoparticle Formulation, Palifermin, Panitumumab, Paraplat (Carboplatin), Paraplatin (Carboplatin), Pegaspargase, Pemetrexed Disodium, Platinol-AQ (Cisplatin), Platinol (Cisplatin), Raloxifene Hydrochloride, Revlimid (Lenalidomide), Rituxan (Rituximab), Rituximab, Sclerosol Intrapleural Aerosol (Talc), Sorafenib Tosylate, Sprycel (Dasatinib), Sterile Talc Powder (Talc), Steritalc (Talc), Sunitinib Malate, Sutent (Sunitinib Malate), Synovir (Thalidomide), Tamoxifen Citrate, Tarabine PFS (Cytarabine), Tarceva (Erlotinib Hydrochloride), Targretin (Bexarotene), Tasigna (Nilotinib), Taxol (Paclitaxel), Taxotere (Docetaxel), Temodar (Temozolomide), Temozolomide, Temsirolimus, Thalomid (Thalidomide), Thalidomide, Totect (Dexrazoxane Hydrochloride), Topotecan Hydrochloride, Torisel (Temsirolimus), Tositumomab and I 131 Iodine Tositumomab, Trastuzumab, Treanda (Bendamustine Hydrochloride), Trisenox (Arsenic Trioxide), Tykerb (Lapatinib Ditosylate), Vectibix (Panitumumab), Velcade (Bortezomib), Vidaza (Azacitidine), Vorinostat, Xeloda (Capecitabine), Zevalin (Ibritumomab Tiuxetan), Zinecard (Dexrazoxane Hydrochloride), Zoledronic Acid, Zolinza (Vorinostat) et Zometa (Zoledronic Acid).

A further subject of the invention is a method for treating or preventing a proliferative disorder or an ocular lesion in a mammal, more particularly a human, comprising the administration of a therapeutically efficient quantity of at least one peptide according to the invention or of at least one chimeric molecule according to the invention, optionally in combination with at least one therapeutic compound useful for the treatment of proliferative disorders, ocular lesions or auto-immune diseases. Said mammal is preferably a mammal suffering from or liable to suffer from a proliferative disorder, an ocular lesion and/or an auto-immune disease.

The invention also concerns a therapeutic composition comprising a peptide or a chimeric molecule according to the invention, and optionally one or more pharmaceutically acceptable excipients.

### Nucleic Acids and Vectors

In one preferred embodiment, the invention concerns the use or administration of a peptide or a chimeric molecule according to the invention.

However, it is also possible to choose to use gene therapy, by using or administering a nucleic acid coding for a peptide of the invention instead of the peptide. In this case, it is administered to the patient a nucleic acid encoding the peptide(s) of interest under conditions such that the peptide(s) are expressed in vivo by the patient's cells into which the nucleic acid has been transferred.

The invention therefore also concerns nucleic acids comprising or consisting of a sequence encoding a peptide of the invention. Said nucleic acids may easily be obtained by cloning fragments of cDNA coding for dermaseptin B2 or the precursor of dermaseptin B2. More particularly, a nucleic acid comprising or coding for a peptide of the invention is represented by the sequence SEQ ID NO: 3.

Such a nucleic acid coding for a peptide of the invention may particularly be in the form of a DNA vector, for example a plasmid vector. It is possible to administer one or more vectors, each vector possibly carrying one or more sequences coding for at least one of the peptides of the invention. In this vector, the sequence(s) coding for at least one of the peptides of the invention are functionally linked to an element or elements allowing expression thereof or regulation of the expression thereof such as transcriptional promoters, activators and/or terminators.

According to one preferred embodiment, a vector is used carrying a sequence coding for the peptide of sequence SEQ ID NO: 1 or SEQ ID NO: 2. According to a more preferred embodiment, a vector is used carrying a nucleic acid of sequence SEQ ID NO: 3.

The DNA vector or vectors may be inserted in vivo using any technique known to persons skilled in the art. In particular, it is possible to insert the DNA vector or vectors in vivo in naked form i.e. without the assistance of any vehicle or system which would facilitate transfection of the vector in the cells (EP 465 529).

A gene gun can also be used, for example by depositing DNA on the surface of “gold” particles and shooting these particles so that the DNA passes through a patient's skin (Tang et al., (1992) Nature 356:152-4). Injections using a liquid gel are also possible to transfect skin, muscle, fat tissue and mammary tissue all at the same time (Furth et al., (1992) Anal Biochem. 205:365-8).

Other available techniques include micro-injection, electroporation, precipitation with calcium phosphate, formulations using nanocapsules or liposomes.

Biodegradable nanoparticles in polyalkyl cyanoacrylate are particularly advantageous. For liposomes, the use of cationic lipids promotes the encapsulation of negatively-charged nucleic acids and facilitates fusion with the negatively-charged cell membranes.

Alternatively, the vector may be in the form of a recombinant virus which, inserted in its genome, comprises a nucleic acid sequence coding for the said peptide(s).

The viral vector may preferably be selected from an adenovirus, a retrovirus, in particular a lentivirus, and an adeno-associated virus (AAV), a herpes virus, a cytomegalovirus (CMV), a vaccine virus, etc. Lentivirus vectors are described for example by Firat et al., (2002) J Gene Med 4:38-45.

Advantageously, the recombinant virus is a defective virus. The term “defective virus” denotes a virus incapable of replicating in a target cell. In general, the genome of defective viruses is devoid of at least the sequences needed for replication of the said virus in the infected cell. These regions can either be eliminated or made non-functional or can be substituted by other sequences and in particular by the nucleic acid which encodes the peptide of interest. Nonetheless, preferably the defective virus maintains the sequences of its genome which are needed for encapsulating the viral particles.

The targeted administration of genes is described for example in application WO 95/28 494.

### Production of Peptides and Chimeric Molecules

The polypeptides useful in the present invention can be synthesized using any method well known to persons skilled in the art. Such methods particularly include conventional chemical synthesis (in solid phase or liquid homogeneous phase), enzymatic synthesis from constitutive amino acids or derivatives thereof, and biological production methods via recombinant host cells.

Synthesis via chemical route is particularly advantageous for reasons of purity, antigen specificity, absence of undesired secondary products and for its easy production. The peptide obtained can then optionally be purified using any method well known to a skilled person. The production method may also comprise one or more steps of chemical or enzymatic modification of the peptide to improve its stability or bioavailability, and one or more steps to bind the peptide to a therapeutic compound.

Synthesis via chemical route includes inter alia synthesis of Merrifield type and Fmoc solid phase peptide synthesis (see for example “Fmoc solid Phase peptide synthesis, a practical approach”, published by W. C. Chan and P. D. White, Oxford University Press, 2000).

The present invention further concerns a method for producing a chimeric molecule according to the invention, comprising:
a) synthesis of a peptide according to the invention, preferably via chemical route,
b) conjugating said peptide with a therapeutic compound useful for treating proliferative disorders, an enzyme capable of converting a molecule into a therapeutic compound useful for treating proliferative disorders, or a carrier protein.

The method for producing a chimeric molecule according to the invention may further comprise a step to formulate the obtained chimeric molecule in a pharmaceutical composition, for example one of the compositions described in the paragraph below.

The peptide of the invention may also be obtained using a biological production method with a recombinant host cell. In such a method, a vector containing a nucleic acid coding for a peptide of the invention is transferred to a host cell which is cultured under conditions allowing the expression of the corresponding peptide.

The peptide produced can then be collected and purified.

The purification methods used are known to a skilled person. The recombinant peptide obtained can be purified from lysates and cell extracts, from the supernatant of the culture medium, using methods performed individually or in combination such as fractionation, chromatographic methods, immunoaffinity techniques using specific mono- or polyclonal antibodies, etc.

The nucleic acid sequence of interest can be inserted into an expression vector in which it is linked functionally to one or more elements allowing its expression or the regulation of its expression, such as transcriptional promoters, activators and/or terminators.

The signals controlling the expression of the nucleotide sequences (promoters, activators, terminating sequences . . . ) are chosen according to the host cell used. For this purpose, the nucleotide sequences of the invention can be inserted in autonomous replication vectors within the chosen host, or integrating vectors of the chosen host. Such vectors are prepared using methods commonly used by skilled persons, and the resulting clones can be inserted in a suitable host using standard methods, such as electroporation for example or precipitation with calcium phosphate.

The cloning and/or expression vectors as described above, containing a nucleotide sequence defined according to the invention are also part of the present invention.

The invention also concerns the host cells transfected transiently or in stable form by these expression vectors. These cells can be obtained by introducing in prokaryote or eukaryote host cells a nucleotide sequence inserted in a vector as defined above, and then culturing said cells under conditions allowing the replication and/or expression of the transfected nucleotide sequence.

Examples of host cells notably include human cells such as HEK293, PER.C6, non-human mammal cells such as CHO, COS, MDCK, insect cells such as SF9 cells, bacteria such as Escherichia coli, strains of fungi and/or yeasts such as L40 and Y90.

### Pharmaceutical Compositions

The present application also describes a pharmaceutical composition comprising as active ingredient at least a peptide or a chimeric molecule according to the invention. In general, said compositions comprise one or more pharmaceutically acceptable excipients.

The peptide and the chimeric molecule of the invention may correspond to any one of the above-described peptides and chimeric molecules. According to one preferred embodiment of the invention, the peptide is a peptide of sequence SEQ ID NO: 1 or SEQ ID NO: 2.

By “excipient” or “pharmaceutically acceptable vehicle” is meant any solvent, dispersion medium, absorption-delaying agents etc., which do not produce any secondary reaction e.g. allergic reaction in human or animal.

Alternatively, the invention also provides a pharmaceutical composition comprising as active ingredient a nucleic acid coding for a peptide of the invention, preferably functionally linked to one or more elements allowing the expression of the peptide or the regulation of its expression, with one or more pharmaceutically acceptable excipients. A preferred pharmaceutical composition comprises a nucleic acid encoding a peptide of sequence SEQ ID NO: 1 or SEQ ID NO: 2.

The invention also provides a pharmaceutical composition comprising at least a peptide, a chimeric molecule or a nucleic acid according to the invention and at least one therapeutic compound useful for treating proliferative disorders, ocular lesions and/or auto-immune diseases, in the presence of one or more pharmaceutically acceptable excipients.

Another embodiment of the invention includes the essentially simultaneous administration of separate compositions comprising on one hand at least a peptide, a chimeric molecule or a nucleic acid according to the invention, and on the other hand at least one therapeutic compound useful for treating proliferative disorders, ocular lesions and/or auto-immune diseases.

Administration may also be performed sequentially using separate compositions comprising on one hand at least a peptide, a chimeric molecule or a nucleic acid according to the invention and on the other hand at least one therapeutic compound useful for treating proliferative disorders, ocular lesions and/or auto-immune diseases.

The dosage evidently depends on the active ingredient under consideration, the mode of administration, the therapeutic indication, the patient's age and condition.

The dose of peptide is preferably 0.1 to 250 mg/kg per day, preferably from 1 to 100 or 0.5 to 100 mg/kg per day, in particular from 0.5 to 5 mg/kg. The unit dose of the peptide preferably contains 12.5 to 200 mg of the peptide.

When the pharmaceutical compositions comprise nucleic acids, the doses of nucleic acid (sequence or vector) to be administered are also adapted according to the mode of administration, to the targeted pathology and the period of treatment notably. In general, when recombinant viruses are used, these are formulated and administered in the form of doses of about 104 to 1014 pfu/ml, preferably 106 to 1010 pfu/ml. The term “pfu” (plaque forming unit) corresponds to the multiplicity of infection of a viral solution and can be determined by infecting a suitable cell culture and by measuring, generally after 48 hours, the number of plaques of infected cells. The techniques for determining the pfu titer of a viral solution are well described in the literature.

The pharmaceutical compositions of the invention can be formulated so that they can be administered to a patient via a single route or via different routes.

The pharmaceutical compositions of the invention may for example be administered via parenteral route, in particular via intravenous, subcutaneous or intramuscular route, via oral route, via inhalation, or via topical or ocular application.

When administration via parenteral route is envisaged, more particularly by injection, the compositions of the invention containing the active ingredient(s) are in the form of solutes and suspension for injection packaged in ampoules or bottles for slow infusion. Injection may particularly be given via sub-cutaneous, intramuscular or intravenous route.

Preferably, in particular for a solid tumour, the pharmaceutical composition may be injected into the tumour.

For administration via oral route, the compositions of the invention are in the form of capsules, effervescent tablets, coated or non-coated pills, sachets, sugar-coated tablets, drinkable ampoules or solutes, micro-granules or sustained release forms.

The forms for parenteral administration are obtained conventionally by mixing the active ingredient(s) with buffers, stabilizing agents, preserving agents, solubilising agents, isotonic agents and suspending agents. In accordance with known techniques, these mixtures are then sterilized and packaged in the form of solutions for intravenous injection.

As buffer, a skilled person may use buffers containing organic phosphate salts.

Examples of suspending agents encompass methylcellulose, hydroxyethylcellulose, hydroxypropylcellulose, acacia and sodium carboxymethylcellulose.

Also, stabilizers useful according to the invention include sodium sulfite and sodium metasulfite, whilst mention may be made of sodium p-hydroxybenzoate, ascorbic acid, cresol and chlorocresol as preserving agents. For the preparation of oral solutions or suspensions, the active ingredients are dissolved or suspended in a suitable vehicle with a dispersing agent, a humecting agent, a suspending agent (e.g. polyvinylpyrrolidone), a preserving agent (such as methylparaben or propylparaben), a taste corrector or flavouring agent.

For the preparation of microcapsules, the active ingredients are combined with suitable diluents, suitable stabilizers, agents promoting the sustained release of active substances or any other type of additive for the formation of a central core which is then coated with a suitable polymer (e.g. a water-soluble resin or water-insoluble resin). Techniques known to those skilled in the art are used for this purpose.

The microcapsules thus obtained are then optionally formulated in suitable dosage units.

Administration via ocular route can also be envisaged, in particular for treatment of an ocular lesion.

The pharmaceutical composition of the invention is then in the form of an ophthalmic composition for local administration to the eye, for example as eye lotion or ophthalmic ointment.

The ophthalmic composition may be an aqueous solution comprising distilled water, a physiological saline solution, in which the peptides of the invention are dissolved. A certain number of additives can be incorporated in the ophthalmic composition if necessary, for example buffer agents, agents ensuring isotonicity with tears, preserving agents, thickeners, stabilizers, anti-oxidants, pH-adjusting agents, chelating agents, etc.

The eye drops are prepared by aseptic handling or sterilization is performed at a suitable step of the preparation.

Ophthalmic ointments can be prepared aseptically by mixing the active ingredient with a usual base. The bases for ophthalmic ointments are for example: vaseline, jelen 50 or plastibase, macrogol, etc. Surfactants can be added to increase hydrophily. Additives such as those described above, for example preserving agents can be added if necessary.

In general, for local ophthalmic application, a satisfactory effect is obtained in adults by administering one droplet to the eye of a preparation containing 0.001 to 10%, preferably 0.01 to 1% weight/volume of the compound of the invention or a pharmaceutically acceptable salt thereof, preferably one to six times per day, each time preferably with one to four droplets in the eye, and if an ophthalmic ointment is used a preparation containing 0.001 to 10%, preferably 0.01 to 1% weight/volume of the compound of the invention or a pharmaceutically acceptable salt thereof is applied to the eye preferably one to six times per day.

The peptides, chimeric molecules and nucleic acids of the invention can also be formulated in the form of liposomes. Liposomes are formed from phospholipids which are dispersed in an aqueous medium and spontaneously form multi-lamellar, concentric, twin-layer vesicles. These vesicles generally have a diameter of 25 nm to 4 μm and can be sonicated leading to the formation of smaller unilamellar vesicles of diameter from 200 to 500 Å, whose core contains an aqueous solution.

The liposomes may be particularly advantageous for administering the medicament product to a precise cell or tissue target. For this purpose, the lipids can be chemically coupled to targeting molecules, such as targeting peptides (e.g. hormones) or antibodies.

The following examples and figures illustrate the invention without limiting the scope thereof.

### DESCRIPTION OF THE FIGURES

FIG. 1A displays histograms showing the number of prostatic adenocarcinoma cells PC-3 (number indicated along the Y-axis x 104) per well according to the concentration of dermaseptin B2 (in μg/ml) used in the treatments. **: p<0.01 versus control (0). ***: p<0.001 versus control (0).

FIG. 1B displays histograms showing the number of mouse embryo cells CES (number indicated along the Y-axis×104) per well according to the concentration of dermaseptin B2 (in μg/ml) used in the treatments. *: p<0.05 versus control (0). ***: p<0.001 versus control (0).

FIG. 10 displays histograms showing the number of prostatic hyperplasia cells G1947 (number indicated along the Y-axis×104) per well according to the concentration of dermaseptin B2 (en μg/ml) used in the treatments. *: p<0.05 versus control (0).

FIG. 2A displays histograms showing the number of human lymphoma cells LB-EBV (number indicated along the Y-axis×104) per well according to the concentration of dermaseptin B2 (in μM) used in the treatments. **: p<0.01 versus control (C). ***: p<0.001 versus control (C).

FIG. 2B displays histograms showing the number of human lymphoma cells Raji (number indicated along the Y-axis×104) per well according to the concentration of dermaseptin B2 (in μM) used in the treatments. ***: p<0.001 versus control (C).

FIG. 3A displays histograms showing the number of colonies of human adenocarcinoma cells PC-3 per mm3 treated with 5 μM of dermaseptin B2 or non-treated (C). ***: p<0.001 versus control (C).

FIG. 3B displays histograms showing the number of colonies of human carcinoma cells MBA-MB231 per mm3 treated with 5 μM dermaseptin B2 or non-treated (C). ***: p<0.001 versus control (C).

FIG. 4A displays a graph showing the changes over time in tumour volume (in mm3) of tumours obtained by xenografts of PC-3 cells on athymic mice. The mice were treated with PBS (▪), Taxol () or dermaseptin B2 (▾), the treatments starting one week after injection of the cells. The arrows indicate the different dosages of the treatment with dermaseptin B2.

FIG. 4B displays a graph showing the weight (in g) of tumours obtained by xenografts of PC-3 cells on athymic mice, after 29 days' treatment. The mice were treated with PBS (▪), Taxol (▴) or dermaseptin B2 (▾), the treatments starting one week after injection of the cells.

FIG. 5A displays histograms showing the number of ABAE cells (percentage) per well according to the concentration of dermaseptin B2 (en μM) used in treatments. ***: p<0.001 versus control (0).

FIG. 5B displays histograms showing the number of capillaries per field formed by ABAE cells in the presence of FGF-2 treated with 5 μM of dermaseptin B2 or non-treated (C). ***: p<0.001 versus control (C).

FIG. 6 displays histograms showing the percentage of PC-3 cells incubated in the absence (C) or presence (D) of 2.5 μM of dermaseptin B2 for 24 h or 72 h, positive both to labelling with Annexin V and to labelling with propidium iodide.

FIG. 7 displays histograms showing the percentage of PC-3 cells incubated in the absence (C) or presence (D) of 2.5 μM of dermaseptin B2 for 24 h or 72 h, negative both to labelling with Annexin V and to labelling with propidium iodide.

FIG. 8 displays histograms showing the percentage of PC-3 cells incubated in the absence (C) or presence (D) of 2.5 μM of dermaseptin B2 for 24 h or 72 h, positive to labelling with Annexin V but negative to labelling with propidium iodide.

FIG. 9 displays histograms showing the percentage of PC-3 cells incubated in the absence (C) or presence (D) of 2.5 μM of dermaseptin B2 for 24 h or 72 h, negative to labelling with Annexin V but positive to labelling with propidium iodide.

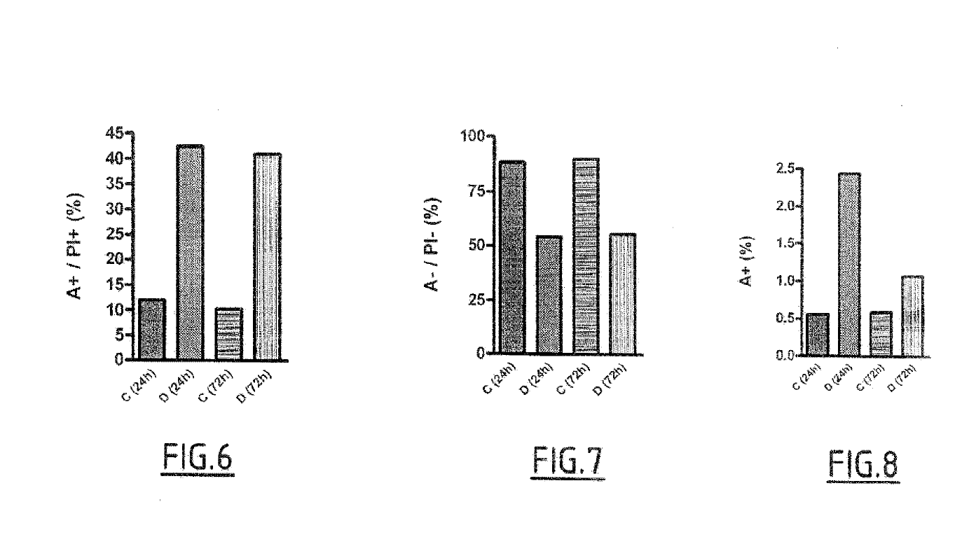

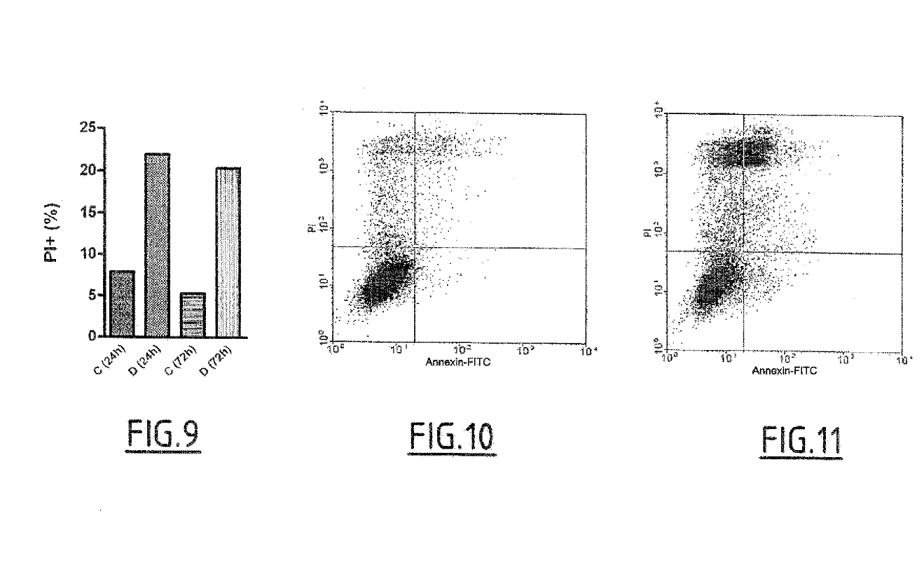

FIG. 10 displays Dot Blot flow cytometry analysis of non-treated PC-3 cells labelled with Annexin V FITC and with propidium iodide (PI).

FIG. 11 displays Dot Blot flow cytometry analysis of PC-3 cells treated for 24 h with 2.5 μM dermaseptin B2, labelled with Annexin V FITC and with propidium iodide (PI).

### EXAMPLES

The following examples show the capability of dermaseptin B2 to inhibit cell proliferation and hence the advantage of its use in the treatment of proliferative disorders.

### Example 1

### Dermaseptin B2 Inhibits the Growth of Adherent or Non-Adherent Tumour Cells but only has Little Effect on Non-Tumour Cells

The anti-proliferative activity of dermaseptin B2 was assessed in vitro on different types of cells: PC-3 adenocarcinoma cells and G1947 human prostatic hyperplasia cells, and on mouse embryo cells (CES). The different types of cells were seeded in 24-well plates at 104 cell/cm2 in 0.5 ml of culture medium (RPMI supplemented with 5% foetal calf serum for the PC-3 and G1947 cells, or DMEM supplemented with 10% foetal calf serum for the CES cells). After 24 hours, the cells were treated with different doses of dermaseptin B2. The treatment was then renewed at days 3 and 5 after seeding of the cells. On the 6th day, an estimated cell count was performed by staining with crystal violet. The cells were rinsed with PBS, fixed on plastic by dehydration with absolute ethanol, and then stained with a 0.2% solution of crystal violet in 2% ethanol for 15 min. After washing, the cells were solubilised with 1% SDS solution. The optical density (OD) of the crystal violet was measured using a spectrophotometer at 595 nm. A standard range was produced for conversion of OD-number of cells.

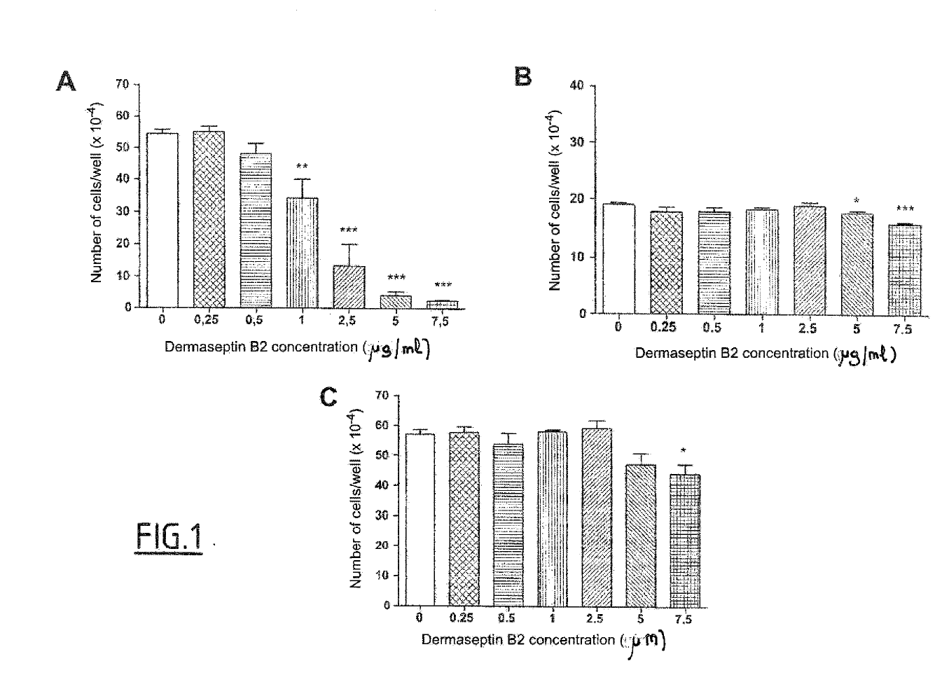

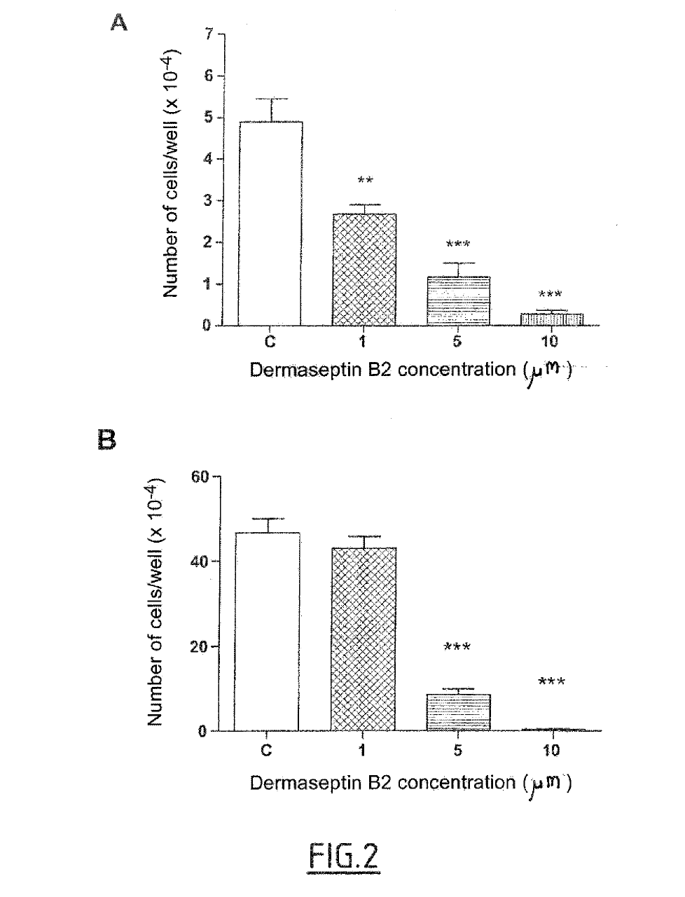

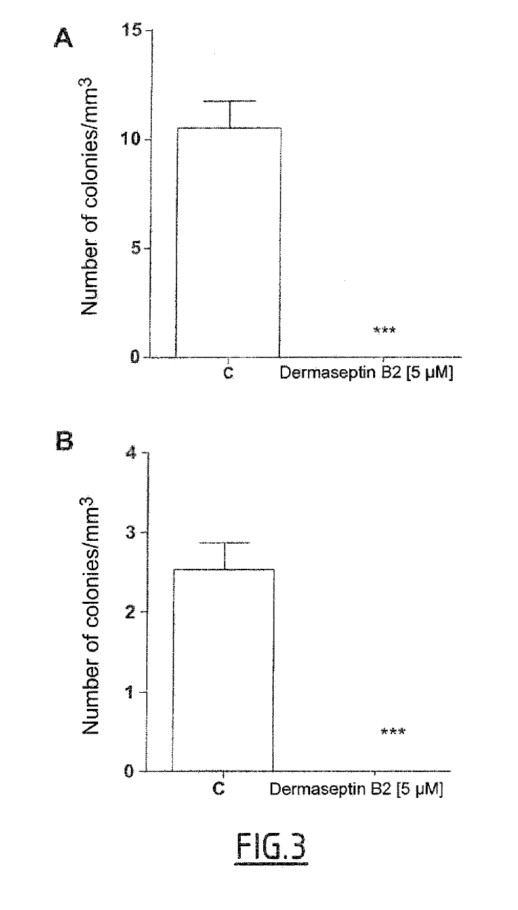

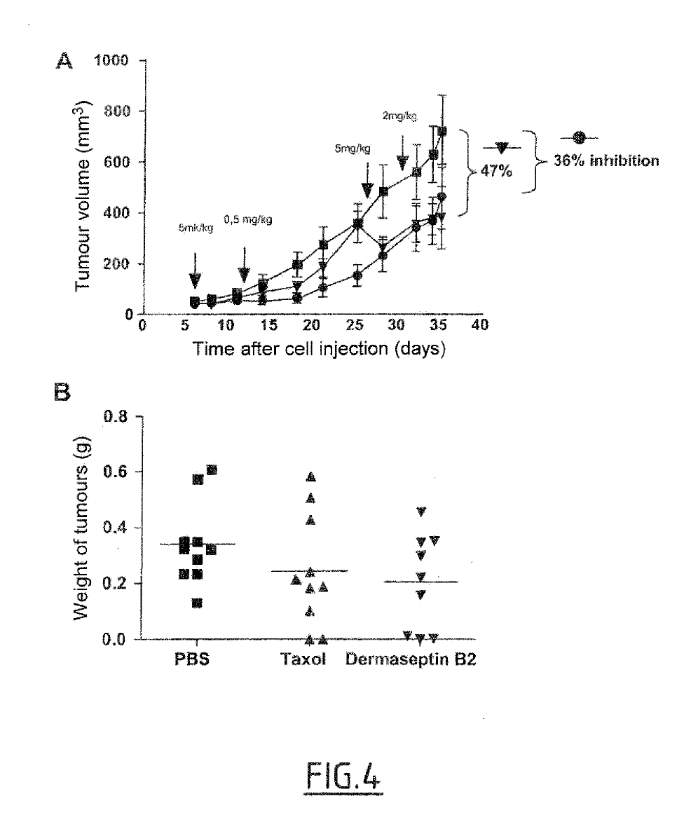

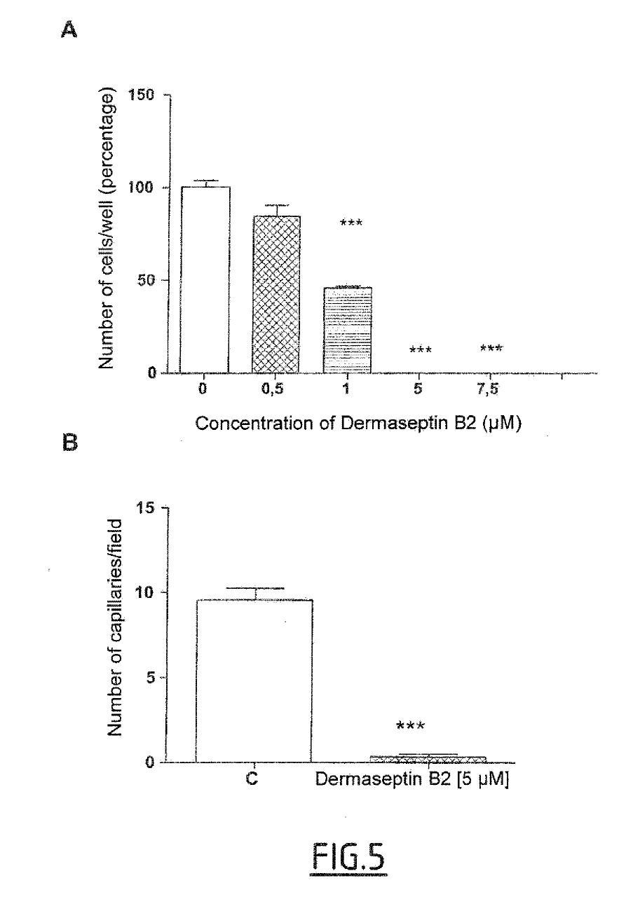

Dermaseptin B2 is capable of inhibiting in dose-dependent manner the proliferation of PC-3 tumour cells (FIG. 1). This inhibition is greater than 90% on and after 5 μM of dermaseptin B2. At this concentration, the effect of dermaseptin B2 is rather more the reflection of a cytotoxic effect on these cells. The inventors effectively observed that the majority of cells at these concentrations were detached from the bottom of the culture dish at the end of the assay. A slight inhibition of about 20% was observed on human G1947 hyperplasia cells with 7.5 μM of dermaseptin B2. This inhibition was even lower (about 10%) on the CES cells with the maximum dose tested. It is to be noted that unlike the PC-3 cells, no cell detachment was observed during treatment for the embryo and G1947 cells.

Dermaseptin B2 was also tested on the growth of two non-adherent lines derived from human B-lymphoma: the Raji and LB-EBV lines. The cells were seeded at 104 cells per well in 0.5 ml of RPMI medium supplemented with 2.5% of decomplemented foetal calf serum. 24 hours after seeding, the cells were treated with different concentrations of dermaseptin B2. The testament was then renewed at days 3 and 5 after seeding the cells. On the 7th day, the cell count was estimated by staining with crystal violet as described previously.

Dermaseptin B2 also inhibits in dose-dependent manner the proliferation of the two human lymphoma lines examined (FIG. 2). At 10 μM dermaseptin B2 inhibits of about 95% the growth of LB-EBV and Raji cells.

### Example 2

### Dermaseptin B2 Inhibits the Growth of Tumour Cells Cultured In Vitro in Soft Agar

To confirm the inhibition of the cell growth observed on plastic with the PC-3 cells, the inventors tested the effect of dermaseptin B2 on the ability of the PC-3 cells to multiply independently of anchoring by forming colonies in agar. For this assay, the cells were seeded at a density of 2.5×10³ cells par cm² diluted in complete culture medium (RPMI supplemented with 5% foetal calf serum) containing 0.35% agar and varying concentrations of dermaseptin B2, in 12-well dishes containing 1 ml of solidified 0.6% agar. The same variable concentrations of dermaseptin B2 were also added to the complete culture medium deposited on top of the cultures on the day of seeding, and twice per week. After incubation for 12 days at 37° C. in an incubator saturated with water vapour and containing 7% CO₂, the colonies having a diameter larger than 50 μm were counted. Each measurement was performed in triplicate and each experiment was repeated three times.

At a concentration of 5 μM, dermaseptin B2 fully inhibits growth of PC-3 cells on soft agar (FIG. 3A). On day 5, in the wells treated with dermaseptin B2, no cell subsisted.

This inhibition of proliferation reflects an effect of dermaseptin B2 on induced cell death or a cytotoxic effect at the doses used. As early as the third day after seeding thereof, the cells treated with dermaseptin do not appear to be refringent when observed under a phase contrast microscope, as do the non-treated cells. When this experiment was conducted with a line of human mammary carcinoma MBA-MB231 cells, similar results were observed (FIG. 3B)

### Example 3

### Dermaseptin B2 Inhibits the Tumour Growth of PC-3 Cells In Vivo in a Nude Mouse Model

Having established that dermaseptin B2 has the capacity to inhibit the growth of PC-3 and MDA-MB231 cells on agar, the inventors tested the effect of this peptide on the growth of tumours induced by injection of PC-3 to nude mice. Batches of 10 nude mice (nude/nude, Laboratoire IFFA CREDO) were injected with 2×106 PC-3 cells. One week after injection of the cells, the animals having a palpable tumour were randomly distributed per cage to be treated by injection into the tumour with 100 μl per day of PBS solution (control batch) or with a solution of dermaseptin B2 diluted in PBS. Dermaseptin B2 was injected at a concentration of 5 mg/kg the first week of treatment, then this dose was reduced to 0.5 mg/kg for 12 days. Subsequent to resumed tumour growth in some treated animals, dermaseptin B2 was again injected at 5 mg/kg for one week and the dose reduced to 2 mg/kg until the end of the treatment. As control, a batch of mice was also treated with Taxol® (paclitaxel, Bristol-Myers Squibb) twice per week via intra-peritoneal injection at 10 mg/kg. The size of the tumours was measured using a calliper twice a week up until sacrifice of the animals 29 days after the beginning of treatment. After sacrifice, the tumours were removed and weighed.

The results are given in FIG. 4A and indicate that dermaseptin B2 induces 47% inhibition of tumour growth compared with the non-treated tumours. This inhibition is substantially higher than observed with Taxol® for which it was only 36%.

After sacrifice of the animals, the tumours were taken and weighed. FIG. 4B shows the difference in weight of the tumours taken from the control animals and the animals treated either with dermaseptin B2 or with Taxol. The results obtained confirm those obtained by measurements of tumour volumes during the treatments, in particular full disappearance of the tumour in 3 mice treated with dermaseptin B2.

### Example 4

### Dermaseptin B2 Inhibits the Growth of Endothelial Cells and the Formation of Pseudo-Capillaries In Vitro

The angiostatic activity of dermaseptin B2 was assessed for its capacity to inhibit firstly the proliferation of adult bovine aortic endothelial cells (ABAE) on plastic and secondly the formation of pseudo capillaries by these same cells in collagen gel. For the proliferation test the ABAE cells were seeded in 24-well plates at a density of 104 cells/well in DMEM medium supplemented with 10% foetal calf serum and 5 ng/ml of FGF-2. As for the previously described proliferation tests, 24 h after seeding the cells were treated with different doses of dermaseptin B2. The treatment was then renewed at days 3 and 5 after seeding the cells. On the 7th day the cell count was estimated by staining with crystal violet.

The inventors have shown that dermaseptin B2 induces dose-dependent and full inhibition of the growth of ABAE cells (FIG. 5A). As for the PC-3 cells, the effect obtained appears to be related to cytotoxicity or induced cell death by dermaseptin B2 on ABAE cells since for the doses of 5 and 7.5 μM, the inventors observed an increase in the presence of dead cells (cell detachment observed during treatment).

The effect of dermaseptin B2 on the formation of pseudo capillaries was assessed through use of the Montessano test. This test allows evaluation of the differentiation of ABAE cells to pseudo capillaries when seeded in a monolayer on collagen 1 gel. For this assay, ABAE cells were seeded in 24-well plates on a monolayer of collagen 1 to the proportion of 105 cells/well in DMEM medium supplemented with 10% foetal calf serum. 24 hours after seeding, 20 ng/ml of FGF-2 were added to the cells in the absence or presence of varying concentrations of dermaseptin B2. This treatment was renewed for two days and the formation of a network of pseudo capillaries was assessed 24 hours later by observation under phase contrast microscope measuring the number of capillaries formed per field of observation (FIGS. 5C and D).

FIG. 5B shows that dermaseptin B2 used here at 5 μM strongly inhibits the formation of pseudo capillaries induced by FGF-2. It is to be noted that, contrary to the proliferation assay, dermaseptin B2 did not have any cytotoxic effect on the monolayer of ABAE cells.

All the results given indicate that dermaseptin B2 is capable of inhibiting two essential steps of angiogenesis: proliferation and differentiation of the endothelial cells.

### Example 5

### Dermaseptin B2 Increases Apoptosis and Necrosis in Human PC-3 Adenocarcinoma Cells

During the proliferation assays of PC-3 or ABAE cells, in the presence of dermaseptin B2, the rapidly observed inhibitor effect and the non-refringence of the treated cells leads to assuming that dermaseptin B2 could have a cytotoxic role or cell death inducing role rather than blocking the growth of sensitive cells. To determine whether dermaseptin B2 could induce apoptosis of sensitive cells, the inventors conducted double labelling experiments with Annexin V and propidium iodide on PC-3 cells in culture on plastic and treated or not treated with dermaseptin B2. These experiments were followed by flow cytometry analysis of the labelled cells.

For this purpose, the cells were seeded in 6-well plates in the absence or presence of 2.5 μM of dermaseptin B2 for 24 or 72 hours. The cells were then detached, washed with cold PBS and re-suspended in 100 μl of fixing buffer (MACS, Miltenyi Biotec) containing Annexin V FITC for 10 min in the dark. After washings in PBS, the cells were again labelled by incubation in a fixing buffer containing a final concentration of 1 μg/ml of propidium iodide (PI) for 5 min in the dark. After further washings the cells were analyzed by passing through a flow cytometer (FACScan, Becton Dickinson Labware) (FIGS. 10 and 11).

Treatment for 24 h of the PC-3 cells with 2.5 μM of dermaseptin B2 induced a strong increase (about 30%) in the cells labelled twofold with Annexin V and PI, translating an increase in cell apoptosis (FIG. 6). This percentage does not change if the cells are treated for 72 h. Conversely, the percentage of non-labelled cells dropped suddenly in the same proportions (FIG. 7). Dermaseptin B2 also causes an increase 5 times the percentage of cells only labelled with Annexin V after 24 h treatment, thereby representing the cells entering into early apoptosis (FIG. 8). Finally, dermaseptin B2 also induces an increase 3 times the percentage of cells labelled solely with PI, thereby translating rapid cell death synonymous with necrosis (FIG. 9). Here again few changes were observed between a treatment time of 24 and 72 h.

These results show that the effect of dermaseptin B2 on PC-3 cells translates as a strong increase in cell death after a treatment time of 24 h.

1. Isolated peptide comprising or consisting of a sequence of amino acids selected from the group consisting of:
the sequence of dermaseptin B2, the sequence of the precursor of dermaseptin B2;
a sequence of amino acids having at least 80% identity with the sequences of dermaseptin B2 or the precursor of dermaseptin B2; and
a fragment of these sequences;
provided that the isolated peptide inhibits cell growth and/or proliferation,
for use in the treatment or prevention of a proliferative disorder, an ocular lesion or an auto-immune disease.

2. The peptide for its use according to claim 1, said peptide comprising or consisting of a sequence of amino acids selected from the group consisting of:
sequences SEQ ID NO: 1 and SEQ ID NO: 2;
a sequence of amino acids having at least 80% identity with sequences SEQ ID NO: 1 or SEQ ID NO: 2; and
a fragment of these sequences;
provided that the isolated peptide inhibits cell growth and/or proliferation.

3. The peptide for its use according to claim 1, said peptide consisting of a sequence of amino acids selected from the group consisting of sequences SEQ ID NO: 1 and SEQ ID NO: 2.

4. The peptide for its use according to claim 1, said peptide comprising a chemical modification improving its stability and/or its bioavailability.

5. A pharmaceutical composition comprising a peptide as defined in claim 1.

6. A chimeric molecule comprising at least one peptide as defined in claim 1, wherein the said peptide is linked to:
a) a therapeutic compound useful for the treatment of proliferative disorders;
b) an enzyme capable of converting a molecule into a therapeutic compound useful for the treatment of proliferative disorders; or
c) a carrier molecule.

7. A pharmaceutical composition comprising a chimeric molecule according to claim 6.

8. The chimeric molecule according to claim 6 for use in the treatment or prevention of a proliferative disorder, of an ocular lesion and/or of an auto-immune disease.

9. The peptide for its use according to claim 1 and a chimeric molecule for its use in the treatment or prevention of a proliferative disorder, of an ocular lesion and/or of an auto-immune disease, wherein said proliferative disorder is selected from solid tumours, leukaemia, tumour metastasis, benign tumours, haemangiomas, rheumatoid arthritis and hepatocellular adenoma.

10. A method for producing a chimeric molecule as defined in claim 6, comprising:
a) the synthesis of a peptide via chemical route; and
b) the conjugation of said peptide with a compound selected from:
(i) a therapeutic compound useful for the treatment of proliferative disorders;
(ii) an enzyme capable of converting a molecule into a therapeutic compound useful for the treatment of proliferative disorders; and
(iii) a carrier protein.

11. The method according to claim 10, further comprising the formulation of said chimeric molecule in a pharmaceutical composition.

12. A nucleic acid comprising or consisting of a sequence coding for a peptide as defined in claim 1, for use in the treatment or prevention of a proliferative disorder, an ocular lesion or an auto-immune disease.

13. A vector comprising a nucleic acid according to claim 12, wherein said nucleic acid is functionally linked to one or more elements allowing the expression of said peptide, for use in the treatment or prevention of a proliferative disorder, an ocular lesion or an auto-immune disease.

14. The nucleic acid for its use according to claim 12 and a vector for its use wherein said nucleic acid is functionally linked to one or more elements allowing the expression of said peptide, for use in the treatment or prevention of a proliferative disorder, an ocular lesion or an auto-immune disease, and wherein said proliferative disorder is selected from solid tumour, a leukaemia, tumour metastasis, benign tumours, haemangioms, rheumatoid arthritis and hepatocellular adenoma.

15. A pharmaceutical composition comprising a nucleic acid as defined in claim 12 and a vector wherein said nucleic acid is functionally linked to one or more elements allowing the expression of said peptide, for use in the treatment or prevention of a proliferative disorder, an ocular lesion or an auto-immune disease.

16. The composition according to claim 5 further comprising a second therapeutic compound useful for the treatment of proliferative disorders, ocular lesions or auto-immune diseases.
